(19) United States
(12) Patent Application Publication
(10) Pub. No.: US 2019/0350939 A1
(43) Pub. Date: Nov. 21, 2019

(54) CANCER TREATMENT METHOD USING Ni-SOD MIMIC COMPOUND

(71) Applicant: NATIONAL CHIAO TUNG UNIVERSITY, Hsinchu City (TW)

(72) Inventors: YUN-MING WANG, KAOHSIUNG CITY (TW); WAY-ZEN LEE, KEELUNG CITY (TW); YU-JEN CHEN, KAOHSIUNG CITY (TW); SHOU-CHENG WU, LUODONG TOWNSHIP (TW); CHIEN-WEI CHIANG, MIAOLI CITY (TW)

(21) Appl. No.: 16/127,806

(22) Filed: Sep. 11, 2018

(30) Foreign Application Priority Data
May 17, 2018 (TW) 107116823

(51) Int. Cl. A61K 31/555; A61K 9/00; A61P 35/00

(52) U.S. Cl. CPC A61K 31/555; A61P 35/00; A61K 9/0019

(57) ABSTRACT

A cancer treatment method using a Ni-SOD mimic compound is provided, which includes administrating the Ni-SOD compound to a cancer cell of a cancer. The structure of the Ni-SOD mimic compound is represented as follows:

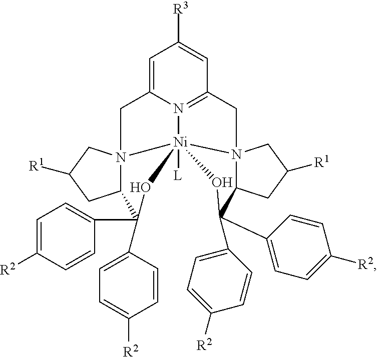

wherein R¹ represents H or A-R′; A represents a bond, O or N; L represents acetonitrile, water, or t-butyl isocyanate; R′ represents H, unsubstituted or substituted alkyl, polyalkoxy, polydimethylsiloxane, polyurethane or other polymer materials or amino acid groups; R² represents unsubstituted or substituted alkyl, alkoxy, siloxy, amino, alkylamine or hydrocarbyl groups; R³ represents unsubstituted or substituted amino, alkylamine, oxyalkylamine groups or magnetic nanoparticles attached oxyalkylamine. Ni is bivalent or trivalent.

### CROSS-REFERENCE TO RELATED APPLICATION

This application claims priority from Taiwan Patent Application No. 107116823, filed on May 17, 2018 at Taiwan Intellectual Property Office, the disclosures of which are hereby incorporated by reference in their entirety for all purposes.

### BACKGROUND OF THE INVENTION

### 1. Field of the Invention

The present invention relates to a superoxide dismutase mimics technical field, and more particularly, to a cancer treatment method using a nickel superoxide dismutase (Ni-SOD) mimic compound.

### 2. Description of the Related Art

Reactive oxygen species (ROS) are side products generated by the organism metabolism, which are widely presented in organisms and have high activity. Hence, ROS play a very important role in cell message delivering and homeostasis. In general, examples of ROS are such as hydrogen peroxide (H₂O₂), hypochlorous acid (HClO), hydroxyl radical (HO.), and superoxide radical (O2⁻.).

The effects caused by ROS in an organism's metabolism have already been fully described. According to prior literature, ROS may not only regulate the cell apoptosis pathway, it may also induce initiative defenses of genes and the start up the ion transmission system. For example, a blood platelet may convene more blood platelets to a wound position so as to repair the wound and to maintain blood balance in vivo. However, the DNA, proteins and cell membrane lipids in the body may be attacked when ROS is in excess and thereby generating oxidation stress, which may affect all of the functions of —SH group contained molecules and —SH groups joined mechanisms, such as proteins and DNA. The oxidation stress may also cause the disruption of message transmission, damage to cell membranes and cellular ion communication and further induce the peroxidation of cell membrane lipids, thereby causing bodily dysfunction and disease.

Generally, when ROS are in excess, the human body may prevent itself from being damaged by ROS through in vivo free radical removing systems, such as alpha-1-microglobulin, superoxide dismutase (SOD), catalases, lactoperoxidases, glutathione peroxidases and peroxiredoxins. Wherein, SOD may remove the superoxide radical and prevent the damage caused by reactive oxygen such that SOD plays an important role in the inhibition of geriatric diseases and the prevention of aging. According to the said properties of SOD taken in conjunction of the contents published by Huang P. et al. in 2000, large amount of O₂⁻. is presented in tumor cells in which the SOD activity is inhibited, which represents that the etiology of tumor is highly possible to be excess ROS. Thus, providing a high SOD concentration condition to tumor cells may be an effective solution for cancer therapy. However, the production and preservation of natural SOD has difficulties in manufacturing processes and cost. Further, if artificial SOD mimics are going to be used in cancer treatment, then those with lower toxicity must be carefully selected to avoid additional damage to the human body. In the existing prior art, the Taiwan Patent Publication No. I449699B disclosed a Ni-SOD mimic as effective antioxidants or free radical scavengers, and further disclosed that the Ni-SOD mimic is appropriated to be utilized in health food or cosmetics, which shows its great biocompatibility. However, there is still a lack of a cancer treatment method using the Ni-SOD mimic.

### SUMMARY OF THE INVENTION

To overcome said problems, the present invention utilizes the Ni-SOD in cancer treatment based on its SOD-like properties, of which the Ni-SOD is taught to be mass synthesized according to the Taiwan Patent Published No. I449699B. The Ni-SOD is able to induce apoptosis pathway and kill tumor cells, which has a potential to replace the efficacy of said nature SOD because of its simple manufacturing process and low cost.

In an aspect of the present invention, a cancer treatment method using a Ni-SOD mimic compound is provided. The method includes: administrating the Ni-SOD mimic compound to a cancer cell of a cancer, wherein the Ni-SOD mimic compound has a structure represented by Formula (I) as follows:

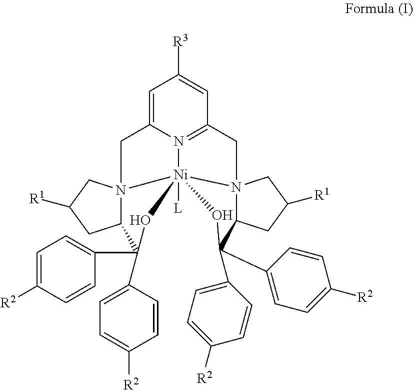

wherein R¹ denotes H or -A-R′; A represents a bond, O or N; L represents acetonitrile, water, or t-butyl isocyanate; R′ represents H, a substituted or unsubstituted alkyl group, a substituted or unsubstituted polyalkoxy group, a substituted or unsubstituted polydimethylsiloxane group, polyurethane, polymer materials or amino acid groups; R² represents a para-substituent of a phenyl ring, and the para-substituent of the phenyl ring is selected from a group consisting of a substituted or unsubstituted alkyl group, a substituted or unsubstituted alkoxy group, a substituted or unsubstituted siloxy group, a substituted or unsubstituted amino group, a substituted or unsubstituted alkylamine group, and a substituted or unsubstituted hydrocarbyl group; R³ represents H or a para-substituent of a pyridine, and the para-substituent of the pyridine is selected from a group consisting of a substituted or unsubstituted amino group, a substituted or unsubstituted alkylamino group, a substituted or unsubstituted oxyalkylamine group, and a substituted or unsubstituted oxyalkylamine group attached to a magnetic nanoparticle; and the Ni is bivalent or trivalent.

Preferably, the Ni-SOD mimic has a structure represented by Formula (II) or (III) as follows:

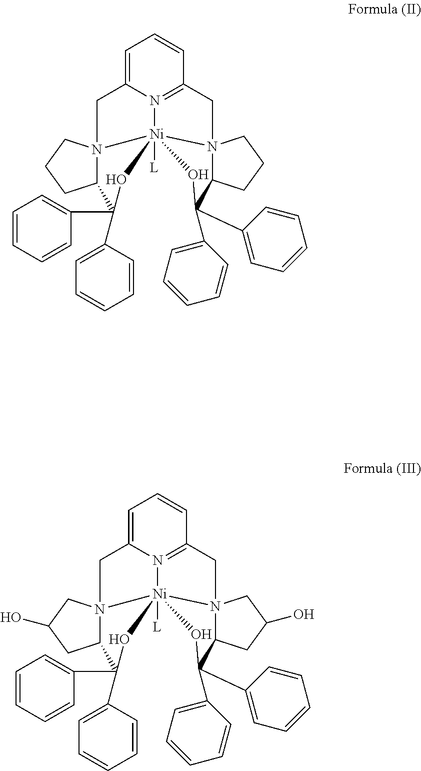

Preferably, the cancer is selected from a group consisting of pancreatic cancer, colorectal cancer, prostate cancer, lung adenocarcinoma, and breast cancer.

Preferably, the administrating is performed with a dose of 0.17 mg/kg-0.37 mg/kg.

Preferably, the frequency of administration is once per day to twice per day.

Preferably, the administration is performed for a period of 18 days.

Preferably, the administration is performed by intravenous injection, intramuscular injection, subcutaneous injection, or a combination thereof.

Preferably, the host of the cancer is selected from a human or a rodent.

Preferably, the Ni-SOD induces an apoptosis pathway.

In summary, the present invention utilizes the Ni-SOD mimic compound in cancer treatment. The anti-cancer drug manufactured by the Ni-SOD mimic compound is able to induce apoptosis pathway and to kill tumor cells thereby achieving excellent treating efficacy. In comparison with prior arts, the Ni-SOD mimic is definitely a potential candidate using in cancer treatment.

### BRIEF DESCRIPTION OF THE DRAWINGS

The patent or application file contains at least one drawing executed in color. Copies of this patent and/or patent application publication with color drawing(s) have been provided to the Office upon request and payment of the necessary fee has been submitted.

FIG. 1 is a block diagram of the cancer treatment method using the Ni-SOD mimic compound.

FIGS. 2A and 2B are histograms representing the survival rate of the cells cultured with the Ni-SOD mimic compound of the present invention.

FIG. 3 represents the analysis results of the cancer cells treated with the Ni-SOD mimic compound of the present invention obtained by a flow cytometry and the photographs captured by a microscope.

FIGS. 4A and 4B are the fluorescent stained microscopy photographs of the cancer cells treated with the Ni-SOD mimic compound of the present invention, wherein blue signal represent nucleus, and green signals represent caspase 9.

FIG. 5 represents the analysis result of the regulation of the cell apoptosis pathway related protein within the cancer cells by the Ni-SOD mimic compound of the present invention.

FIG. 6 represents the analysis result of the regulation of the cell apoptosis pathway related protein within the cancer cells by the Ni-SOD mimic compound of the present invention.

FIG. 7 shows the tumor size measurement results of the cancer mice models treated with the Ni-SOD mimic compound of the present invention by photographs.

FIG. 8 represents the quantization results of the measurement of FIG. 7 and the chart representing weight change of the cancer mice models.

FIGS. 9A and 9B are the H&E stained results of the slice of the tumor position of the cancer mice model treated with the Ni-SOD mimic compound of the present invention.

FIGS. 10A and 10B are the H&E stained results of the slice of the organs of the cancer mice model treated with the Ni-SOD mimic compound of the present invention.

### DESCRIPTION OF THE PREFERRED EMBODIMENTS

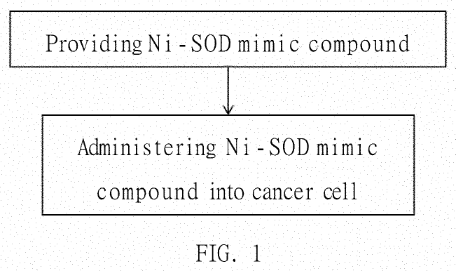

In an embodiment of the present invention, a cancer treatment method using a Ni-SOD mimic including the steps of administrating the Ni-SOD mimic compound to the cancer cell of a cancer is provided, as shown in FIG. 1.

Herein, a cancer treatment method using a Ni-SOD mimic compound is provided and described in detail.

In particular, the Ni-SOD mimic compound has a structure represented by Formula (I) as follows:

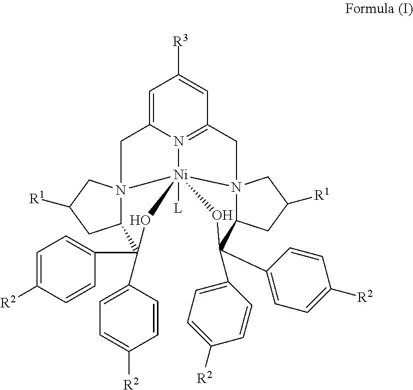

wherein R¹ denotes H or -A-R′; A represents a bond, O or N; L represents acetonitrile, water, or t-butyl isocyanate; R′ represents H, a substituted or unsubstituted alkyl group, a substituted or unsubstituted polyalkoxy group, a substituted or unsubstituted polydimethylsiloxane group, polyurethane, polymer materials or amino acid groups; R² represents a para-substituent of a phenyl ring, and the para-substituent of the phenyl ring is selected from a group consisting of a substituted or unsubstituted alkyl group, a substituted or unsubstituted alkoxy group, a substituted or unsubstituted siloxy group, a substituted or unsubstituted amino group, a substituted or unsubstituted alkylamine group, and a substituted or unsubstituted hydrocarbyl group; R³ represents H or a para-substituent of a pyridine, and the para-substituent of the pyridine is selected from a group consisting of a substituted or unsubstituted amino group, a substituted or unsubstituted alkylamino group, a substituted or unsubstituted oxyalkylamine group, and a substituted or unsubstituted oxyalkylamine group attached to a magnetic nanoparticle; and the Ni is bivalent or trivalent.

Preferably, the Ni-SOD mimic compound may be the Wct003 having the structure represented by Formula (II) or the Wct006 having the structure represented by Formula (III). The preparation procedures of Wct003 and Wct006 are described below.

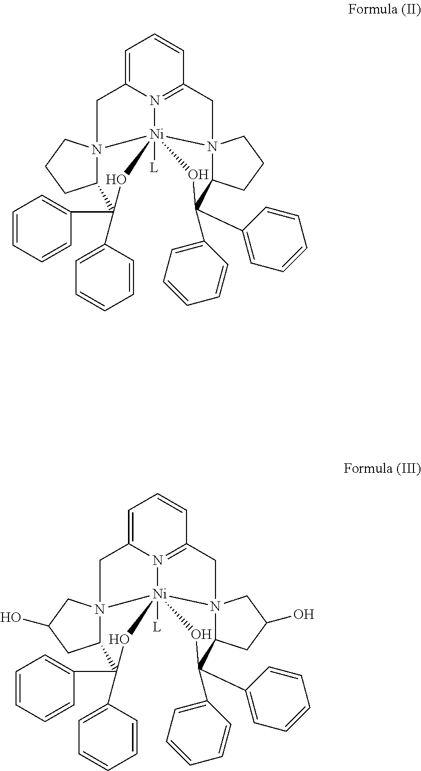

Preparation of Wct003

In an embodiment, the preparation methods of Ni-SOD mimic compounds Wct003 having the structure represented by Formula (II), Wct006 having the structure represented by Formula (III) and the derivatives thereof are disclosed in Taiwan Patent Published No. I449699B. The detail of the preparation method and process can be found in said Patent document. In particular, said patent provides a Ni contained complex or the derivatives thereof for mimicking the activity center of a Ni-SOD so as to become a Ni-SOD mimic compound. The present invention provides a cancer treatment method using said Ni-SOD mimic compound.

In general, the synthesis of the Ni-SOD mimic compound is referred to as Reaction (I). In Reaction (I), [2,6-bis(((S)-2-(diphenylhydroxymethyl)-1-pyrrolidinyl)methyl)pyridine] (H2BDPP) or the derivatives thereof is reacted with [Ni(CH₃CN₆)](ClO₄)₂ alone, or it is reacted with [Ni(CH₃CN₆)](ClO₄)₂ and sodium hydride together.

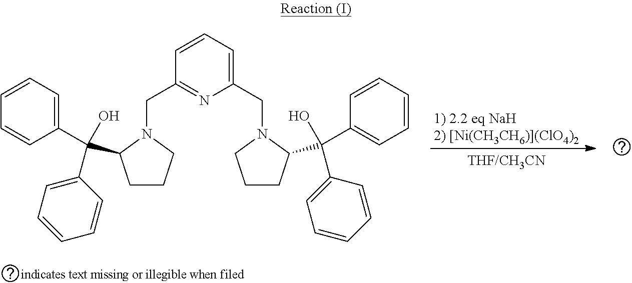 [SMILES: OC(c1ccccc1)(c1ccccc1)[C@@H]1CCCN1Cc1cccc(CN2CCC[C@H]2C(O)(c2ccccc2)c2ccccc2)n1]

For instance, the OH-BDPP of which the hydroxyl group is bonded with a pyrrolidine alkyl group is provided as a reaction precursor. Sequentially, 0.128 g (0.2 mmol) of OH-BDPP is reacted with 0.12 g (0.5 mmol) of sodium hydride and 0.101 g (0.2 mmol) of [Ni(CH₃CN₆)](ClO₄)₂, and then placed at room temperature for 2 hours to obtain a penta-coordinated bivalent nickel complex Ni—OH-BDPP, Ni—OH-BDPP which may further be prepared into two derivatives: penta-coordinated trivalent nickel complex [Ni—OH-BDPP]PF₆ and hexa-coordinated bivalent nickel complex Ni—OH—H₂BDPP (Wct006). Similarly, Wct003 may also be prepared as in the said procedure.

Subsequently, the synthesized Ni-SOD mimic compound may be found to have functions of killing tumor cells by in vitro and in vivo experiments. Wherein, the cell models used for in vitro experiments are human pancreatic cancer cell lines (MiaPaCa-2 and Panc-1), human colorectal cancer cell line (HT29, and Colo205), human prostate cancer cell line (LNCap), human lung adenocarcinoma cell line (A549), and human breast cancer cell line (SKBR3). The models used in in vivo experiments are BALB/c nude mice. Hereinafter, the analysis methods and the results thereof of the in vitro experiments including cell cytotoxicity assay, cell apoptosis assay, cell morphology assay, confocal fluorescent microscopy assay, and cell apoptosis pathway assay; and in vitro assays including living animal analysis, tumor tissue slice analysis, and tissue immunostaining analysis are described in detail.

MTT Cell Proliferation Assay

MTT compound is utilized as a testing agent in the MTT cell proliferation assay. The full name of MTT is 3-(4,5-cimethylthiazol-2-yl)-2,5-diphenyl tetrazolium bromide, which is a yellow color compound. MTT is a dye which is able to receive a hydrion and to be joined in the electron transport chain (ETC) within the mitochondria in a living cell. The tetrazolium ring of MTT may be broken by the effect caused by succinate dehydrogenase (SDH) and cytochrome C, and thereby generating blue crystals. The amount of the generated blue crystals is directly proportional to the amount of living cells (SDH in dead cells are vanished, so that MTT cannot be reduced). By measuring the absorbance peak value of MTT spectrum, the amount of the living cells may be indirectly measured. Hence, MTT cell proliferation assay is an effective index to indicate cell viability.

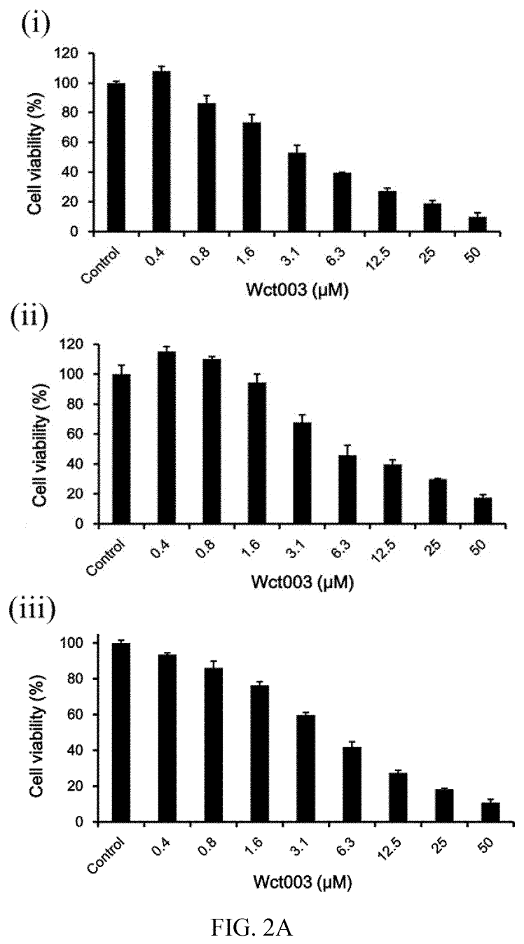

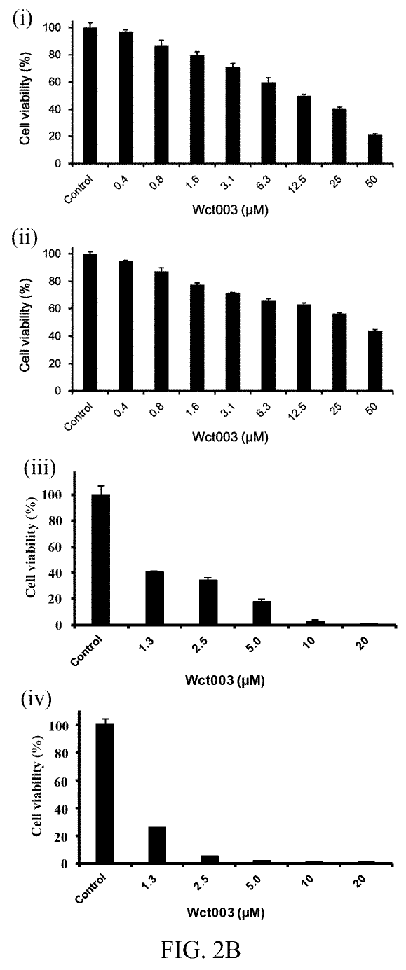

In an embodiment, as shown in FIGS. 2A and 2B, various concentrations (0-50 μm) of Ni-SOD mimic compound (Wct003) are co-cultured with pancreatic cancer cell line MiaPaCa-2 (part (i) of FIG. 2A), pancreas/catheter epithelial cancer cell line Panc-1 (part (ii) of FIG. 2A), human prostate cancer cell line LNCap (part (iii) of FIG. 2A), human lung adenocarcinoma cell line A549 (part (i) of FIG. 2B), human breast cancer cell line SKBR3 (part (ii) of FIG. 2B), human colorectal cancer cell line HT29 (part (iii) of FIG. 2B), and Colo205 (part (iv) of FIG. 2B) for 24 hours. Further, the cytotoxic efficacy of Wct003 is discussed according to the results.

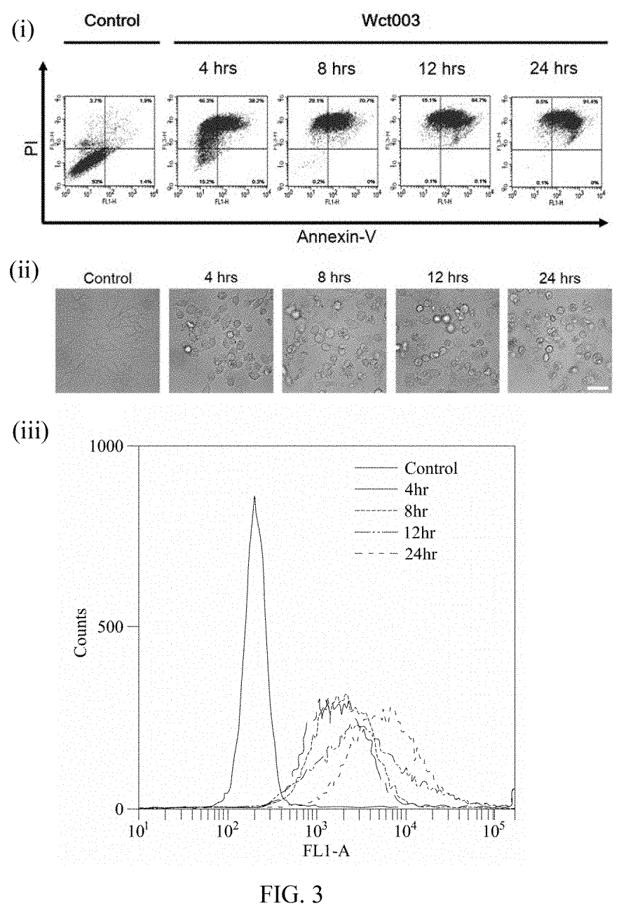

As shown in FIGS. 2 and 3, the cell viabilities of MiaPaCa-2, Panc-1, HCT116 and LNCap co-cultured with Wct003 are all below 20%. The IC₅₀ are 4.7 μM (MiaPaCa-2), 6.1 μM (Panc-1), 0.83 μM (HT29), 0.66 μM (Colo205), 5.6 μM (LNCap), and 11.6 μM (A549) and 27.3 μM (SKBR3). Apparently, Wct003 can effectively inhibit the growing of cancer cells of MiaPaCa-2, Panc-1, LNCap, HT29, and Colo205 cancer cell lines, and the IC₅₀ in those cell lines are all below 10 μM. In summary, MiaPaCa-2, Panc-1, LNCap, HT29, and Colo205 cancer cell lines have the potential to be utilized in a cancer treatment method, and the MiaPaCa-2 may be the most possible one.

Cell Apoptosis and Cellular Morphology Assay

In an embodiment, the fluorescent signals of Annexin-V and propidium iodide (PI) are utilized to define whether apoptosis is induced in the cells. Wherein, Annexin-V is a Ca⁺ dependent phospholipid binding protein (35-36 kDa), which has high affinity to phosphatidylserine (PS). When a cell is in an apoptosis pathway, the PS of cell membrane may flip outside the outer membrane, and the flipped PS may be identified by and combined with Annexin-V, so that the apoptosis pathway may be detected by such proteins. On the other hand, PI may penetrate dead cells and stain the nucleus in a fluorescent red color. Hence, the double staining method using PI and Annexin-V may determine the stage of cell apoptosis in order to define the cell at an early stage or a late stage of cell apoptosis pathway. Moreover, the present embodiment further tags the cells by a caspase 3 antibody and analyzes the cell by a flow cytometry to determine whether apoptosis is induced in the cells. Furthermore, during the aforementioned experiments, photographing by a microscope may be performed simultaneously to observe the change of the cell morphology.

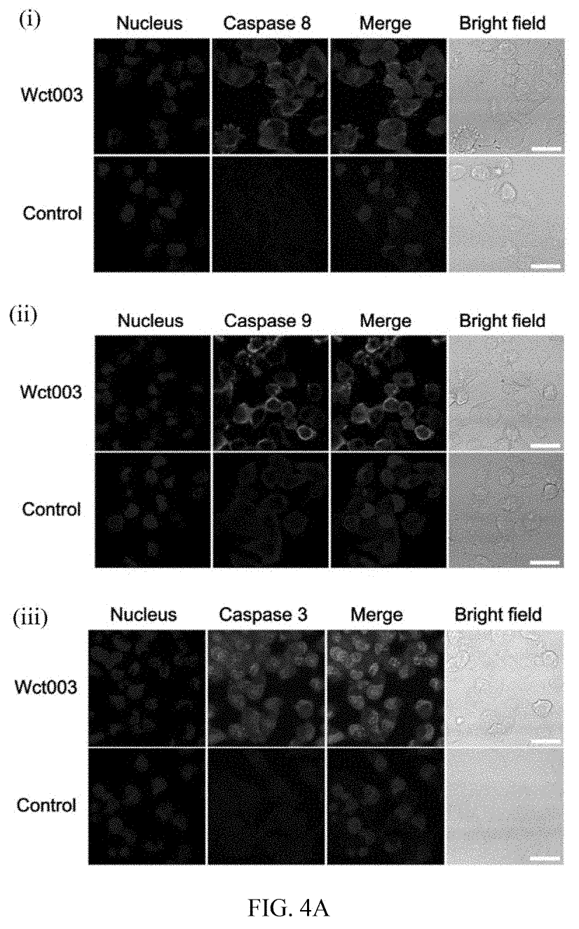

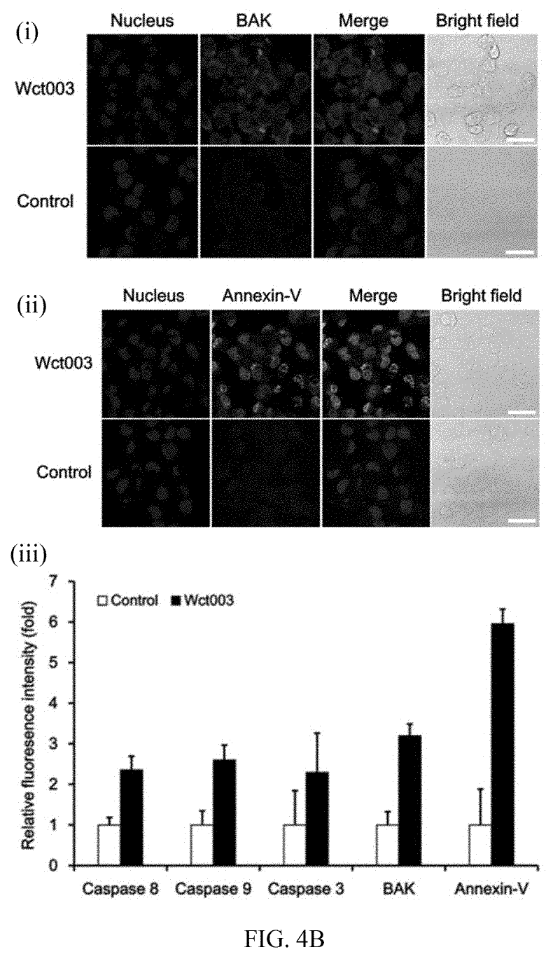

As shown in FIG. 3, the human pancreatic cancer cell line MiaPaCa2 is co-cultured with 4.7 μM of Wct003 for 4, 8, 12 and 24 hours, and analyzed by the Annexin-V/PI double staining method and cell morphology analysis to measure the cytotoxicity of Wct003 against the human pancreatic cancer cell line. Part (i) of FIG. 3 shows the results of the Annexin-V/PI double staining method analysis, wherein the apoptosis induced cell amount percentages are 38.2% (4 hours), 70.7% (8 hours), 84.7% (12 hours), and 91.4% (24 hours) when MiPaCa-2 is co-culture with Wct003. Apparently, more than 50% of cells are in the apoptosis pathway at the 8 hour point, and more than 90% of cells are in the apoptosis pathway at the 24 hour point. Moreover, the microscopy cell morphology images of part (ii) of FIG. 3 also show that the cells generate apoptosomes, wherein the scale of the image is 100 μm. As shown in part (iii) of FIG. 4A, MiCapa-2 co-cultured with Wct003 for 4 hours obviously expressed caspase 3, and the fluorescent signal intensity increased with time. Thus, it can be proven that Wct003 is able to induce apoptosis in cells, and the result is apparent after treatment for 4 hours.

Confocal Fluorescence Microscopy Assay

In an embodiment, the condition of the cells administrated with the Ni-SOD mimic compound of the present invention is observed by a confocal fluorescence microscope taken in conjunction with immunofluorescence staining Sequentially, the tumor cells (about 1×10⁵ cells) are seeded on a cover glass (24 mm×24 mm) and grown for 24 hours. The tumor cells are then co-cultured with Wct003 (4.7 μM) at 37□ for 4 hours. Then, tumor cells are washed by PBS buffer 3 times and are fixed by 4% formaldehyde at room temperature for 30 minutes. Next, 2 mL of primary antibody is added and stay overnight at 4□. On the next day, the cells are washed by PBS 3 times and secondary antibodies are added at room temperature and reacted for 30 minutes, following by PBS washing 3 times. Finally, the cover glass is fixed on a glass slide by a mixture of PBS:glycerol=1:1, and observed by the confocal fluorescence microscope to consider the cell morphology and protein expressions. In the present embodiment, the nucleus is stained by DAPI for immunofluorescence, and the cell apoptosis related proteins are stained by FITC.

In FIGS. 4A and 4B, the blue regions (DAPI) represent the nucleus, and the green regions (FITC) shown in parts (i)-(iii) of FIG. 4A and parts (i) and (ii) of FIG. 4B represent the cell apoptosis related protein including anti-caspase 8, anti-caspase 9, anti-caspase 3, anti-BAK and anti-Annexin-V, wherein the scale bar is 40 μm. The ratios between the expression of the cell apoptosis related proteins of each of the said groups and the control group which only contains cell medium are 2.3 (caspase 8), 3.6 (caspase 9), 2.2 (caspase 3), 3.3 (BAK) and 5.9 (Annexin-V) times respectively.

Apoptosis Pathway Assay

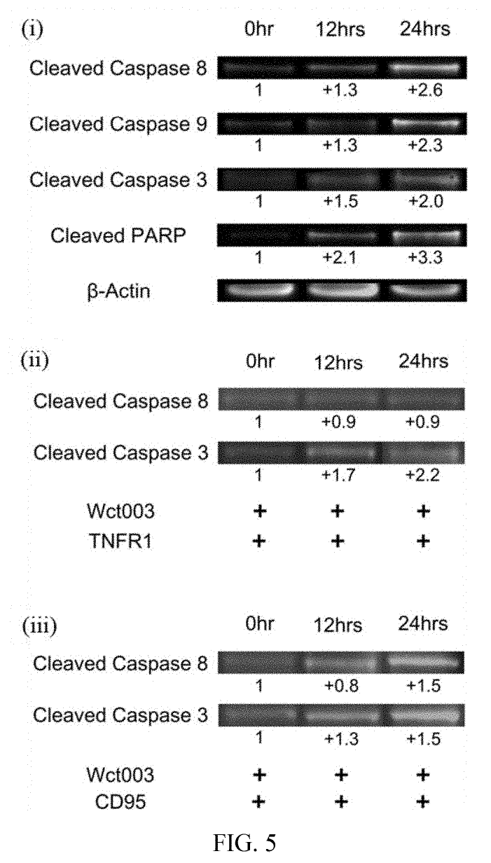

In an embodiment, a method arranged with the Western Blot method can observe whether apoptosis is induced in the cells or not according to the expressions of the apoptosis related proteins. Sequentially, MiaPaCa-2 is seeded to a number of 1×10⁶ and co-cultured with Wct003 (IC₅₀ dose) at 37° C. for 1 hour. The cell proteins are then extracted by RIPA buffer after collection the cells are washed twice. The proteins are separated by 12% of polyacrylamide gel electrophoresis (SDS-PAGE) and transferred by 1.2 mA/cm² of current for 45 minutes in order to blot the proteins into a PVDF membrane (Immobilon™-P Transfer Membrane, Millipore). Next, a coloring analysis is performed after labeling by an antibody. In the present embodiment, the antibodies used for analyzing the cell apoptosis pathway are caspase 3, caspase 8, caspase 9, p-AKT, p-ERK1/2, p-p38 and p-STAT3. The results are shown in FIG. 5.

As shown in part (i) of FIG. 5, caspase 8 is extensively expressed at 24 hours, which is almost 2.6 times in comparison with that at the 0 hour point. Further, caspase 9, caspase 3 and PARP are regulated to extensive express of which the expression level is about 2.3, 2.0 and 3.3 times in comparison with that at the 0 hour point, respectively. Furthermore, parts (ii) and (iii) of FIG. 5 shows the results how TNFR, CD95 antibodies block TNFα and FasL receptors affect the expressions of caspase 9 and caspase 3. As shown in the figure, at the 24 hour point, the caspase 8 protein expression and the caspase 3 protein expression of the TNFR1 treated group are 0.9 and 2.2 times in comparison with that at the 0 hour point, respectively; the caspase 8 protein expression and the caspase 3 protein expression of the CD95 treated group are 1.5 and 1.5 times in comparison with that at the 0 hour point, respectively.

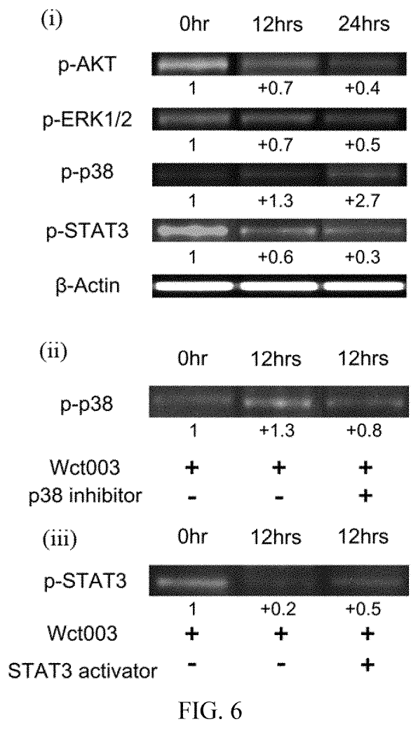

In addition, part (i) of FIG. 6 shows the expression levels of other cell apoptosis related proteins including p-AKT, p-ERL1/2 and p-STAT3. As shown in the figure, at the 12 hour point, the expression levels of the apoptosis-related protein p-AKT, p-ERK1/2 and p-STAT3 have decreased, which are 0.7, 0.7 and 0.6 times in comparison with that at the 0 hour point, respectively; and the expression levels of p-p38 have obviously increased, which are about 1.3 times in comparison with that at the 0 hour point. At the 24 hour point, the trend did not change too much.

Parts (ii) and (iii) of FIG. 6 show the results how treating p38 inhibitor and STAT activator affect the apoptosis related proteins. As shown in the figures, at the 12 hour point, the expression levels of p-p38 and p-STAT3 are 0.8 and 0.5 times in comparison with that at 0 hour point. The aforementioned results prove that Wct003 induces apoptosis in cells.

In Vivo Study

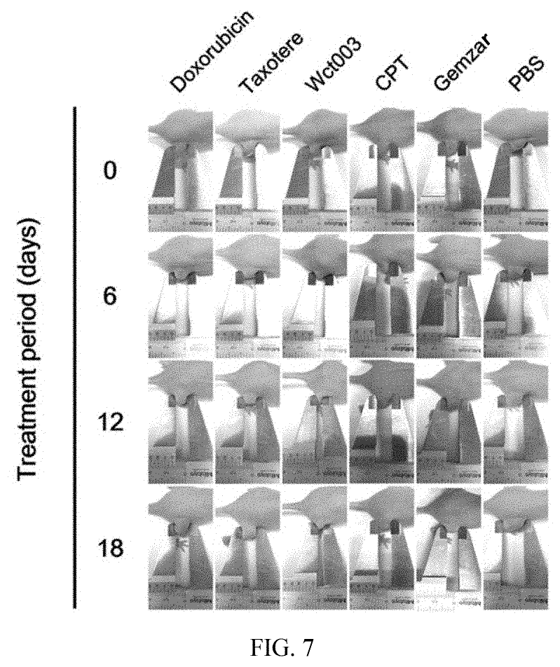

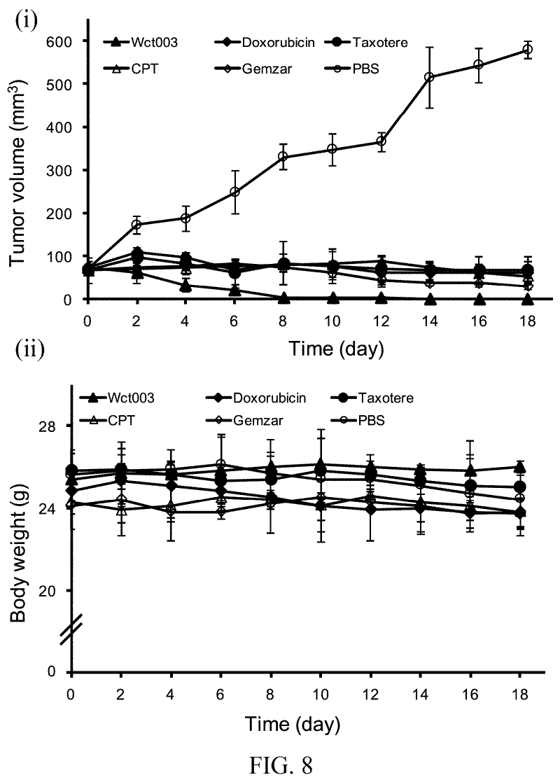

In an embodiment, mice (BALB/cAnN.Cg-Foxn1nu/CrlNarl mice) are reared until 6 weeks of age, and 1×10⁶ of MiaPaCa-2 cells are subcutaneously injected into the right rear leg of the mice. When the tumor has grown to 125 mm³, Wct003 (0.27 mg/kg), the comparative drugs (Doxorubicin (0.27 mg/kg), Taxatere (0.27 mg/kg), CPT (0.27 mg/kg) and Gemzar (0.27 mg/kg)) and control group (PBS) are injected into the mice by tail vein injection, and choose the PBS-injected group as the control group. The experiment is performed until the 18^{th} day, and the tumor size is measured every 2 days. The measurement formula of the tumor size is as follows: ½ (L×W²), wherein L represents the longest diameter of the tumor; W represents the shortest diameter of the tumor. The whole experiment is repeated 3 times and indicated with (Mean±SD), and the results are shown in FIGS. 7 and 8.

As shown in FIG. 7 and part (i) of FIG. 8, both Wct003 and the comparative group drug inhibits the tumor growth during the experiment. However, Wct003 efficiently kills the tumor cells at the 14^{th} day, and the comparative drugs are not able to completely kill all of the tumor cells until the 18^{th} day from the termination date of the experiment. At the 18^{th} day from the termination date of the experiment, the tumor growth conditions are measured. The results of the tumor sizes are 0 (Wct003), 64 (Doxorubicin), 68 (Taxatere), 52 (CPT), and 30 (Gemzar) mm³, and the tumor of the PBS group grows up to 600 mm³. The results show that the group treated with drugs is able to inhibit the tumor growth, wherein the Wct003 has the best efficacy, which completely kills the tumor at the 14^{th} day; the Doxorubicin and Taxatere have the second best efficacy.

In addition, as shown in part (ii) of FIG. 8, during the drug administration period of 18 days, the body weight of all groups of mice does not obviously change. It proves that Wct003 and the comparative drugs do not induce obvious side effects in nude mice.

Tumor Section and Immunohistochemistry Studies

In an embodiment, the aforementioned mice are satisfied after the 18 days of in vivo experiments, and the tumor cells are collected and are soaked in formalin for paraffin embedding slices. By immunofluorescence staining of the tissue of the tumor slices, whether the efficacy of Wct003 kills tumor cells in vivo can be further analyzed. In the present embodiment, H&E staining is provided as an exemplary example to analyze the condition of tumor cells. The antibodies (caspase 9, caspase 3, Bak, PARP, Bcl-XL, Bcl-2 and Bax) target and stain the apoptosis related proteins to shown the expression levels.

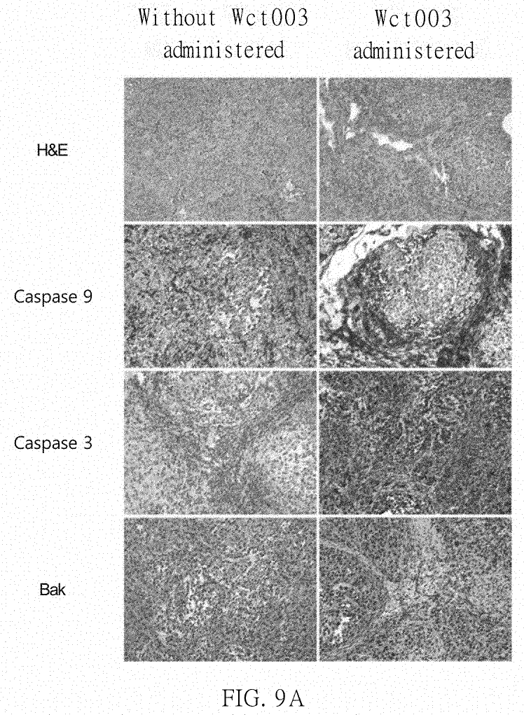

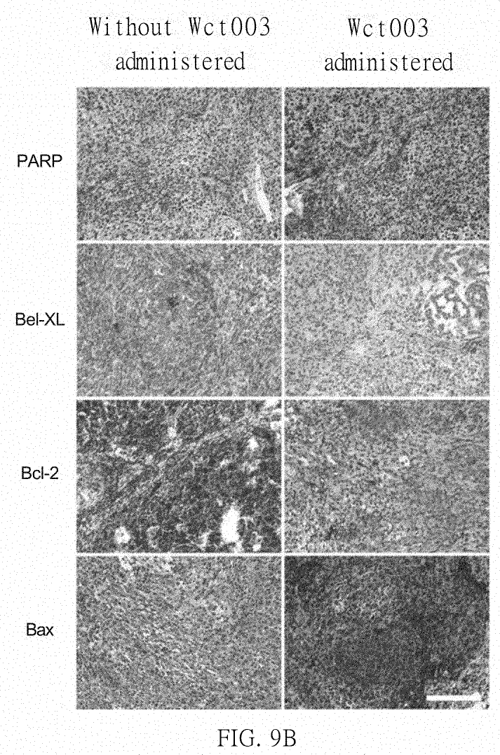

FIGS. 9A and 9B show the staining results of the tumor tissue section slices. As shown in the figure, the slice of the tumor tissue injected with Wct003 shows the signals of apoptosis related proteins caspase 9, caspase 3, BAK, PARP and Bax expression (brown color); and the area of the brown parts of the Bcl-XL AND Bcl-2 injected groups are less than that of Wct03 injected group, which represents that the expression has a reduce tendency. The results show the same conclusion as the aforementioned results about the apoptosis related protein expression levels determined by the confocal fluorescent microscope. Hence, it can be proved that Wct003 induces apoptosis in cells and kills the tumor. In the figures, the scale bar is 100 μm.

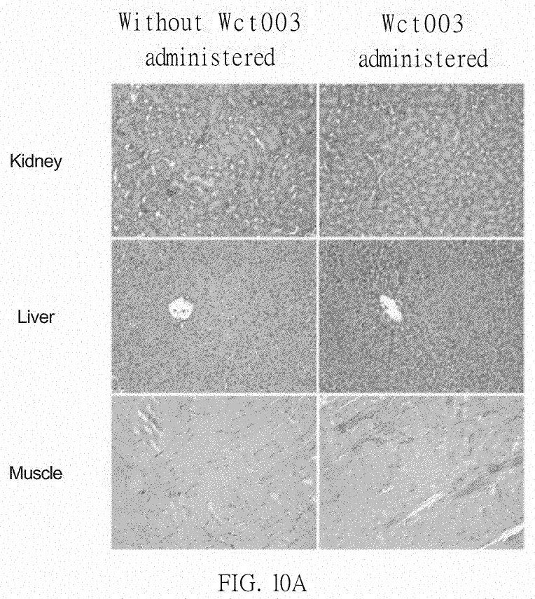

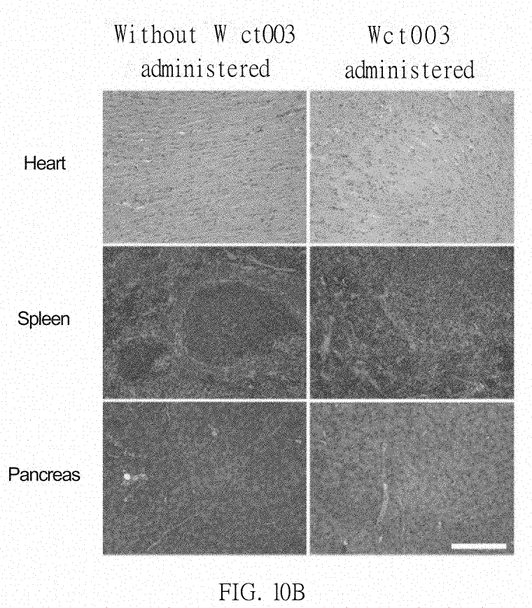

FIGS. 10A and 10B show the staining results of the slices of other organs. As shown in the figures, the condition of the tissue slices of the Wct003 injected nude mice are not obviously different from that of PBS injected nude mice. Hence, it is reasonable to consider that Wct003 is a safe anti-cancer drug which only provides the cytotoxic effects on tumors but does not obviously harm other normal organs. In the figure, the scale bar is 100 μm.

As described above, it has been proven by the in vitro and in vivo experiments that the cancer treatment method using the Ni-SOD mimic compound has an excellent effect. Preferably, the method is not only able to be administered to a rodent but also a human.

Preferably, according to the cell lines utilized in the aforementioned in vitro and in vivo experiments, it is also proven that the cancer treatment method using the Ni-SOD mimic compound performs an excellent effect on pancreatic cancer, colorectal cancer, prostate cancer, lung adenocarcinoma, and breast cancer. Preferably, the administration is performed with a dose of 0.17 mg/kg-0.37 mg/kg, and the frequency of the administration is once per day to twice per day. Preferably, the administration is performed for a period of 18 days.

The present invention utilizes the Ni-SOD mimic compound in cancer treatment. The anti-cancer drug manufactured by the Ni-SOD mimic compound is able to induce apoptosis pathway and to kill tumor cells thereby achieving excellent treating efficacy, which has enough efficacy to replace effects provided by natural SOD. Furthermore, the preparation of the Ni-SOD mimic compound in the present invention has lower cost and simpler procedure in comparison with that of prior arts which producing natural SOD. In addition, the efficacy of the Ni-SOD mimic compound in cancer treatment has been proven by the aforementioned experiments. Hence, the Ni-SOD mimic is definitely a potential candidate for use in cancer treatment.

The content of the embodiment described above is merely at least one of various implementations. A person skilled in the art is able to understand the core concept of the present invention after reading the content of above, and to modify the embodiments depending on requirements. In other words, the embodiments described above are not intended to limit the present invention. The scope protected by the present invention is defined by the appended claims.

1. A method of treating cancer in a subject in need thereof, comprising administering to the subject a Ni-SOD mimic compound of Formula (I): 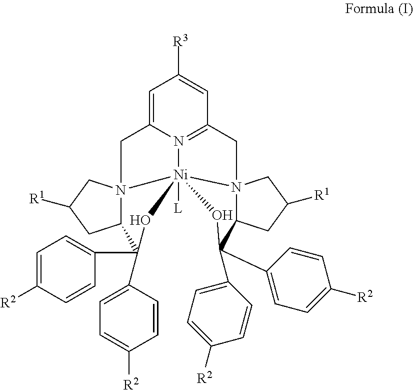
wherein R¹ denotes H or -A-R′;
A is a bond, O or N;
L is acetonitrile, water, or t-butyl isocyanate;
R′ is H, a substituted or unsubstituted alkyl group, polyurethane, or an amino acid groups;
R² is H, a substituted or unsubstituted alkyl group, a substituted or unsubstituted alkoxy group, a substituted or unsubstituted siloxy group, a substituted or unsubstituted amino group, or a substituted or unsubstituted alkylamine group;
R³ represents is H, a substituted or unsubstituted amino group, or a substituted or unsubstituted alkylamino group; and
the Ni is bivalent or trivalent.

2. The cancer treatment method according to claim 1, wherein the Ni-SOD mimic compound has a structure represented by Formula (II) or (III) as follows:

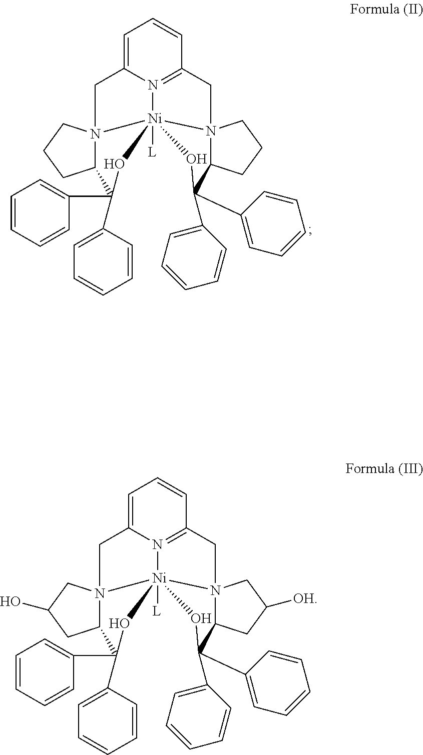

3. The cancer treatment method according to claim 1, wherein the cancer is selected from a group consisting of pancreatic cancer, colorectal cancer, prostate cancer, lung adenocarcinoma, and breast cancer.

4. The cancer treatment method according to claim 1, wherein the administering is performed with a dose of 0.17 mg/kg-0.37 mg/kg.

5. The cancer treatment method according to claim 1, wherein a frequency of the administering is once per day to twice per day.

6. The cancer treatment method according to claim 1, wherein the administering is performed for a period of 18 days.

7. The cancer treatment method according to claim 1, wherein the administering is performed by intravenous injection, intramuscular injection, subcutaneous injection, or a combination thereof.

8. The cancer treatment method according to claim 1, wherein the subject is a human or a rodent.

9. The cancer treatment method according to claim 1, wherein the Ni-SOD mimic compound induces an apoptosis pathway.
